(19) United States
(12) Patent Application Publication
(10) Pub. No.: US 2025/0073458 A1
(43) Pub. Date: Mar. 6, 2025

(54) LEAD WITH IMPROVED MRI COMPATIBILITY

(71) Applicant: Axonics, Inc., Irvine, CA (US)

(72) Inventors: Xuechen HUANG, Irvine, CA (US); Guangqiang JIANG, Irvine, CA (US); Henry LEE, Irvine, CA (US)

(73) Assignee: Axonics, Inc., Irvine, CA (US)

(21) Appl. No.: 18/818,508

(22) Filed: Aug. 28, 2024

Related U.S. Application Data
(60) Provisional application No. 63/535,212, filed on Aug. 29, 2023.

(51) Int. Cl. A61N 1/08; A61N 1/05; A61N 1/36; A61N 1/375

(52) U.S. Cl. CPC A61N 1/086; A61N 1/05; A61N 1/3605; A61N 1/375

(57) ABSTRACT

A system for providing neurostimulation to a patient includes a pulse generator having a housing. An implantable neurostimulation lead is configured to connect with the pulse generator and the pulse generator is configured to generate a plurality of electrical impulses for delivering a neurostimulation treatment to the patient through the lead when the lead is implanted at a target location. The lead includes one or more conductors extending from a proximal end of the lead to one or more neurostimulation electrodes disposed at or near a distal end of the lead. The lead includes an energy absorber including carbon nanotube material and extending substantially along the length of the one or more conductors.

This application discloses an improved neurostimulation lead that has one or more extra electromagnetic shielding layers in the lead body. The disclosed neurostimulation lead includes a shielding or integrated carbon nanotube (CNT) material. The CNT material may be a composite material that may also include any other type of nonmetallic electrically conductive material. Any CNT shielding layer may be further coated with a high-permittivity material such as iron or nickel. The use of an electromagnetic shielding layer allows for improved MRI compatibility of the neurostimulation lead.

A typical neurostimulation lead generally has electrodes at the distal end. On the proximal end, the lead has electrical contacts connected to an implantable pulse generator (IPG) which generates electrical signals. The conductors in the lead provide an electrically conductive path along which electrical signals may be delivered to a neural target for therapeutic purposes. When a patient requires an MRI scan, the entire implanted system (including an IPG and a neurostimulation lead) is exposed to strong electromagnetic (EM) fields generated by an MRI scanner. The lead conductors, acting like antennas, may absorb electromagnetic energies, specifically the radio frequency energy in Mega Hertz frequencies (i.e., 64 MHz or 128 MHz). The absorbed EM energy on the lead may pose high thermal stress to tissue in contact with the electrodes and voltage stress to the IPG device, potentially leading to tissue or device damage. In particular, typical neurostimulation leads dissipate heat at the distal end of the electrodes, creating a point source of heat that may harm the patient. Thus, patients with an implanted system are typically not permitted to have an MRI scan for at least these reasons. Therefore, it is desirable to have a neurostimulation lead with improved electromagnetic shielding to reduce absorbed EM energy and to better dissipate heat.

This application discloses a neurostimulation system including an improved neurostimulation lead that has one or more electromagnetic shielding layers in the lead body. The shielding layer protects the conductors from exposure to electromagnetic energy. The neurostimulation system includes an implantable pulse generator (IPG) with a housing and an improved neurostimulation lead including a set of one or more lead conductors. The neurostimulation system as a whole is designed to conduct electrical pulses generated by the IPG through the neurostimulation lead and to a target location within a patient's body, thereby providing neurostimulation therapy. In one embodiment, the disclosed neurostimulation lead includes at least one shielding layer that includes carbon nanotube (CNT) materials. The reference to CNT throughout this disclosure encompasses both composite CNT materials and homogenous CNT materials. For example, the CNT material disclosed herein may be a composite that also includes any other type of suitable electrically conductive material. Alternatively, the CNT used as a heat sink or shielding layer may be made from a homogenous CNT material. The CNT material may take the form of an elongated cord, yarn, fiber or wire structure. Alternatively, the CNT material may be a sheet, coating or film structure. In addition, in certain disclosed embodiments, the CNT shielding material of the neurostimulation lead may be further coated with a high-permittivity material such as iron or nickel.

The disclosed neurostimulation lead may have multiple forms of CNT shielding, including (but not limited to) shielding layers on individual lead conductors, a shielding layer wrapped around the whole set of lead conductors, and CNT material configured as a fiber like material that is integrated with and extends along the length of the lead conductors. The one or more forms of CNT shielding may be electrically interconnected in order to distribute heat and electromagnetic energy evenly along the structure of the neurostimulator lead. The one or more forms of CNT shielding may further be electrically connected to the housing of the IPG, allowing the housing to receive and dissipate heat and electromagnetic energy.

### BRIEF DESCRIPTION OF THE DRAWINGS

FIG. 1 shows an example of an implantable neurostimulation system designed to provide neurostimulation therapy to a patient.

FIG. 2 shows an example of the distal end of a typical neurostimulator lead with four electrodes.

FIGS. 3A-3C show examples of conductor coils encased in a variety of EM shielding layer designs. FIG. 3A shows an example of a coil with a braided carbon nanotube shield, FIG. 3B shows an example of a coil with a spiral wire/served carbon nanotube shield, and FIG. 3C shows an example of a coil with a foil/spiral tape carbon nanotube shield.

FIG. 4 shows an example of a structure for a neurostimulation lead including one or more conductors and an integrated carbon nanotube shielding wire.

FIG. 5 shows a cross-section of an example of a structure for a neurostimulation lead including one or more conductors with carbon nanotube coating on each conductor.

FIG. 6 shows an example of a structure for a neurostimulation lead with an integrated carbon nanotube shielding wire, a braided shielding layer and a jacket.

FIG. 7 shows a side view of an example of a structure for a neurostimulation lead with carbon nanotube fibers woven in and out of the lead conductors.

FIGS. 8A-8B show examples of weave patterns in which carbon nanotube fibers are woven in and out of the lead conductors.

### DETAILED DESCRIPTION

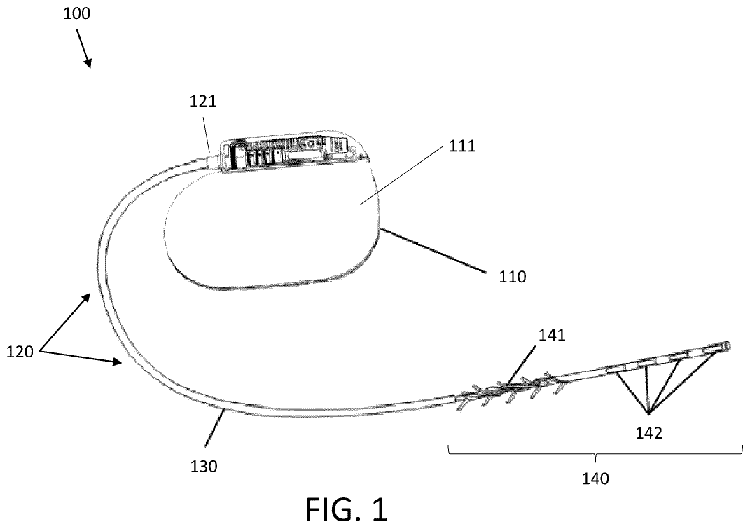

FIG. 1 shows an example of an implantable neurostimulation system 100 designed to provide neurostimulation therapy to a patient. The implantable system 100 includes an implantable pulse generator (IPG) 110 that is coupled to a neurostimulation lead 120 that includes a group of neurostimulation electrodes 142 at a distal end 140 of the lead 120. The IPG 110 may include a header portion and a feed-through assembly coupled to both a lead connector stack located in the header and to internal circuitry located in the main portion of the IPG 110. In some embodiments, a Balseal® connector block is employed and the connector block may be connected to feed-through pins. In some embodiments, the electrode pins are part of a plurality of pins, wherein the remainder of the plurality of pins are not configured for connecting to the connector stack and are instead non-functional, connected to ground, or directly connected to other components such as an antenna.

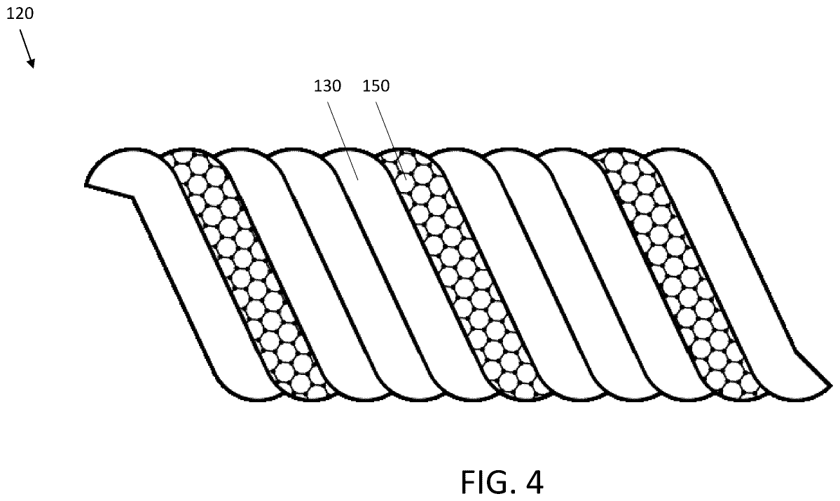

In one embodiment, the IPG 110 is enclosed in a housing 111. In one embodiment, the lead 120 connects with the IPG 110 and the IPG housing 111 by means of a lead connector 121. The lead 120 may include a lead anchor portion 141 with a series of tines extending radially outward so as to anchor the lead 120 and maintain a position of the neurostimulation lead 120 after implantation. In one embodiment, the lead 120 contains one or more conductors 130 (see FIG. 4). In some embodiments, the IPG 110 provides monopolar or bipolar electrical pulses that are delivered to the targeted nerves through one or more neurostimulation electrodes 142, typically four electrodes. In sacral nerve stimulation, the lead 120 is typically implanted through the S3 foramen.

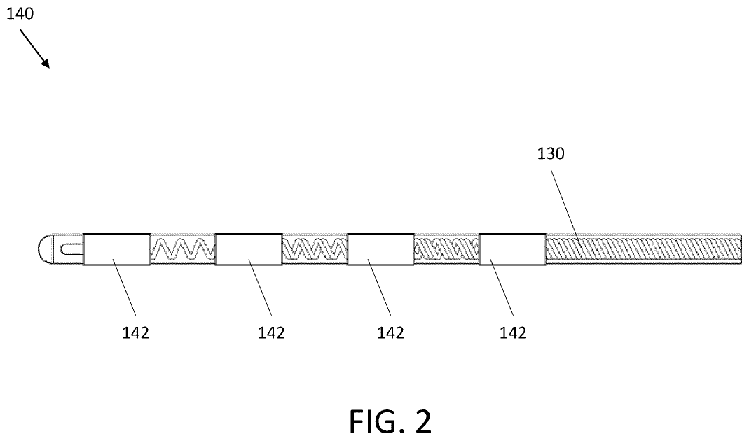

FIG. 2 shows an example of the distal end 140 of a typical neurostimulator lead 120 with four electrodes 142. A typical neurostimulation lead 120 generally has electrodes 142 at the distal end 140. On the proximal end, the lead 120 has electrical contacts connected, by means of a lead connector 121 (see FIG. 1), to an IPG 110 which generates electrical signals. In one embodiment, the lead conductors 130 in the lead 120 provide an electrically conductive path to allow the electrical signals to be delivered to the neural target via the electrodes 142 for therapeutic purposes.

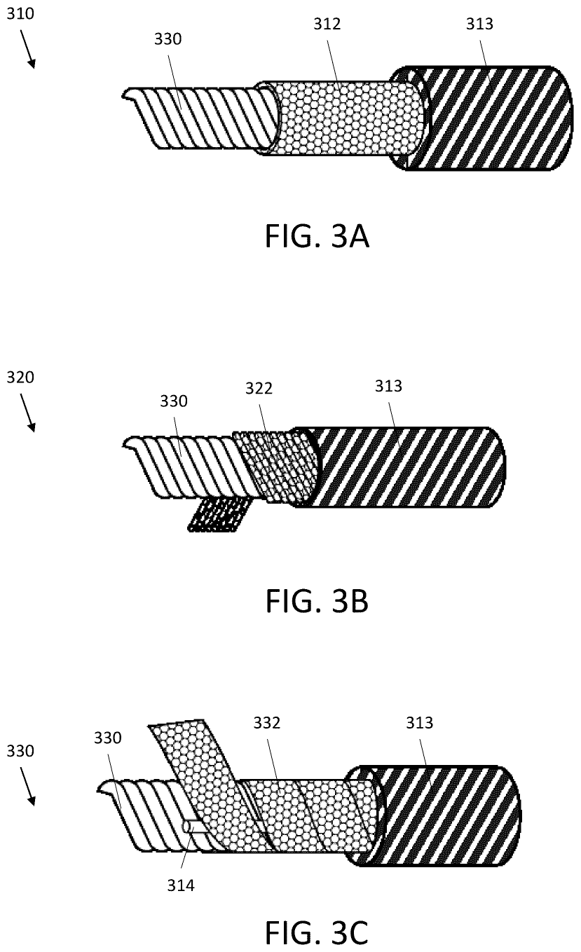

FIGS. 3A-3C show examples of conductor coils 330 encased in a variety of EM shielding layer designs. FIG. 3A shows an example of a conductor coil 330 with a braided CNT shield 312, FIG. 3B shows an example of a conductor coil 330 with a spiral wire/served CNT shield 322, and FIG. 3C shows an example of a conductor coil 330 with a foil/spiral tape CNT shield 332. The braided shield cable 310 includes a conductor coil 330, a braid CNT shield 312, and a jacket 313. The spiral wire/served shield cable 320 includes a conductor coil 330, a spiral wire/served CNT shield 322, and a jacket 313. The foil/spiral tape shield cable 330 includes a conductor coil 330, a spiral wire/served CNT shield 332, a jacket 313, and a drain wire 314.

These EM shields may be composed at least in part of a carbon nanotube (CNT) material. In one embodiment, CNTs are composed of a single atomic layer of carbon in a cylindrical configuration. CNT material can be manufactured by various methods, but are mostly commonly made using chemical vapor deposition. The end result from this process is a paper like, ultra-thin sheet that can be further processed into a variety forms, including but not limited to yarns, sheets, and tapes. Alternatively, CNT material can be made using suspension solutions that can be sprayed or printed on a deposition surface like a regular ink.

Although CNTs are nonmetallic, they can conduct electricity like metals; yet they still have the flexibility, low weight, malleability, and corrosion resistance qualities of polymers. When CNTs are used for electromagnetic shielding, they can be made into structures like a braided shield, served shield, or spiral tape shield (FIGS. 3A-3C). CNT shielding may also be configured in manners other than those disclosed in this application.

FIG. 4 shows an example of a structure for a neurostimulation lead 120 including one or more conductors 130 and an energy absorber 150. The energy absorber 150 (which may be alternatively referred to as a “heat sink”, “energy shield” or an “EM energy absorption conduit”) may be a shielding wire that is integrated with the one or more conductors 130, and the energy absorber 150 may be composed of a material including CNT. This energy absorber 150 may be configured to act as an energy absorber and/or energy conduit for the system as a whole. In one embodiment, the energy absorber 150 conducts absorbed EM energy along the length of the energy absorber 150, avoiding the risks of thermal damage presented by a point-source dissipation by moving the EM energy to a safe dissipation site. In another embodiment, the energy absorber 150 can transfer the absorbed EM energy to the IPG housing 111 (see FIG. 1), wherein the housing 111 additionally acts as an energy dissipater. Due to the high IPG-tissue interface impedance, the IPG housing 111 may dissipate the absorbed EM energy by first converting the absorbed EM energy to thermal energy at the IPG-tissue interface, and then dispersing the heat at the large tissue contact area of the IPG housing. In one embodiment, the energy absorber 150 transfers EM energy to the IPG housing 111 through a lead connector 121 (see FIG. 1).

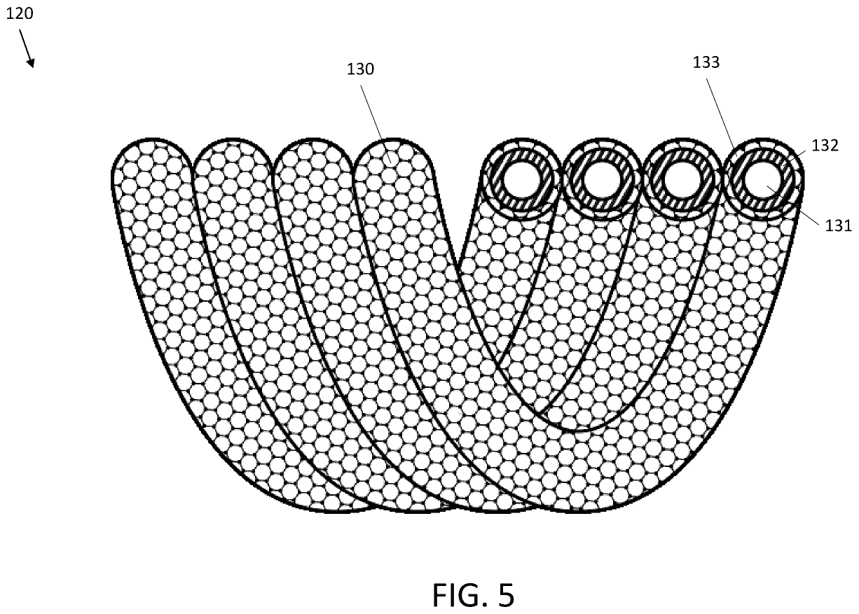

FIG. 5 shows an example of a structure for a neurostimulation lead 120 with CNT coated on each lead conductor 130. Embodiments disclosed herein relate to methods of manufacturing and formation of aforementioned CNT electromagnetic shielding structure/layer 133 in a neurostimulation lead 120. CNT can be made in a liquid suspension. Therefore, it can be used for printing of conductive traces or spraying, like an ink or paint. In some embodiments, the polymer insulation layer 132 for each lead conductor 130 may be coated with a liquid formulation including suspended CNT particles. In one embodiment, this process produces a conformal, conductive, and thin CNT coating layer 133 outside the insulation layer 132, thus providing electromagnetic interference (EMI) shielding for the conductor core 131. Various types and methods can be used to create the conformal CNT coating 133, including (but not limited to) dispersion, immersion, and injection.

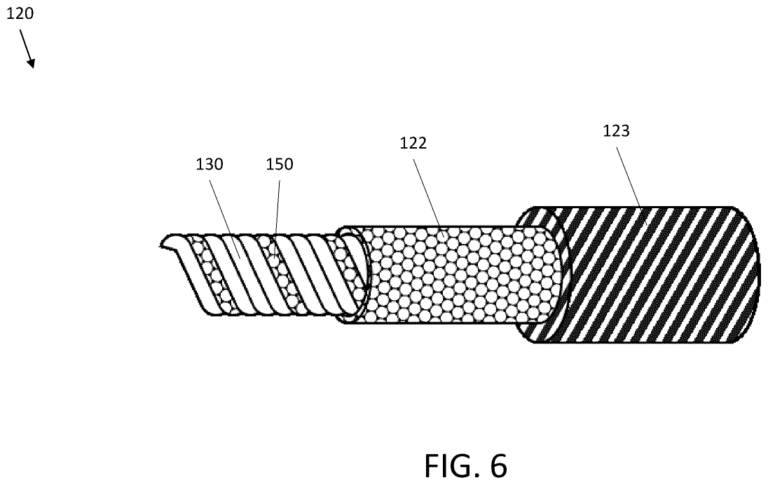

FIG. 6 shows an example of a structure for a neurostimulation lead 120 with an integrated CNT energy absorber 150, a CNT shielding layer 122 and a jacket 123. In one embodiment, the neurostimulation lead 120 has an outer layer polymer jacket 123 forming a long cylindrical lead body 120. In one embodiment, the lead conductors 130 are placed within the lead body 120 and each lead conductor 130 has its own insulated jacket 132 (see FIG. 5). In another embodiment, each lead conductor 130 may further include a CNT coating layer 133 (see FIG. 5). To achieve EMI shielding, the CNT electromagnetic shielding layer 122 can be added into the lead body 120, in between the lead conductors 130 and the outer layer polymer jacket 123. On the proximal end, the CNT shielding layer 122 can be electrically connected to the IPG housing 111 by means of a lead connector 121. In one embodiment, any EM fields can be absorbed by the CNT shielding layer 122 and dissipated through the IPG housing 111. In such an embodiment, the lead conductors 130 inside the shielding layer 122 are shielded from any external EM disturbance. The addition of an integrated CNT shielding wire 150 may also further provide shielding from external EM disturbance while also providing another channel through which to dissipate any absorbed EM energy. To this end, the integrated CNT energy absorber 150 may also be electrically connected to the IPG housing 111 and/or the CNT electromagnetic shielding layer 122. This electrical connection may run through the lead connector 121.

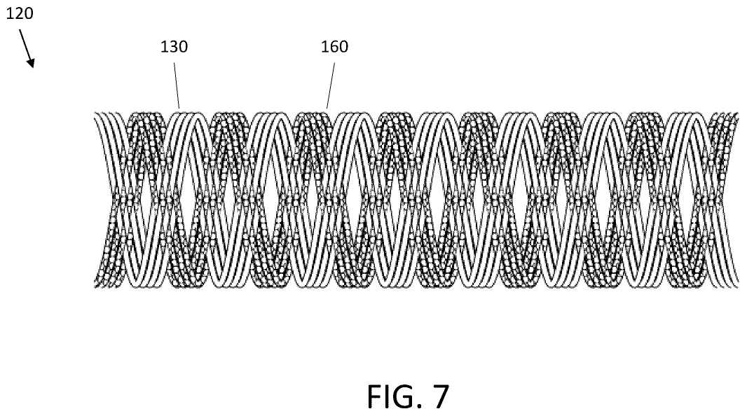

FIG. 7 shows a side view of an example of a structure for a neurostimulation lead 120 with CNT fibers 160 woven in and out of the lead conductors 130. In one embodiment, the lead conductors 130 and one or more CNT fibers 160 may weave together to form a unified coil, thereby comprising the core of the neurostimulation lead 120. The lead conductors 130 and CNT fibers 160 may coil around the length of a shared axis in opposite directions, e.g. the lead conductors 130 may coil with a left-handed thread pattern and the CNT fibers 160 may coil with a right-handed thread pattern. In order to support such coil patterns, the lead conductors 130 and CNT fibers 160 may enmesh with each other at regular intervals along the length of the neurostimulation lead 120.

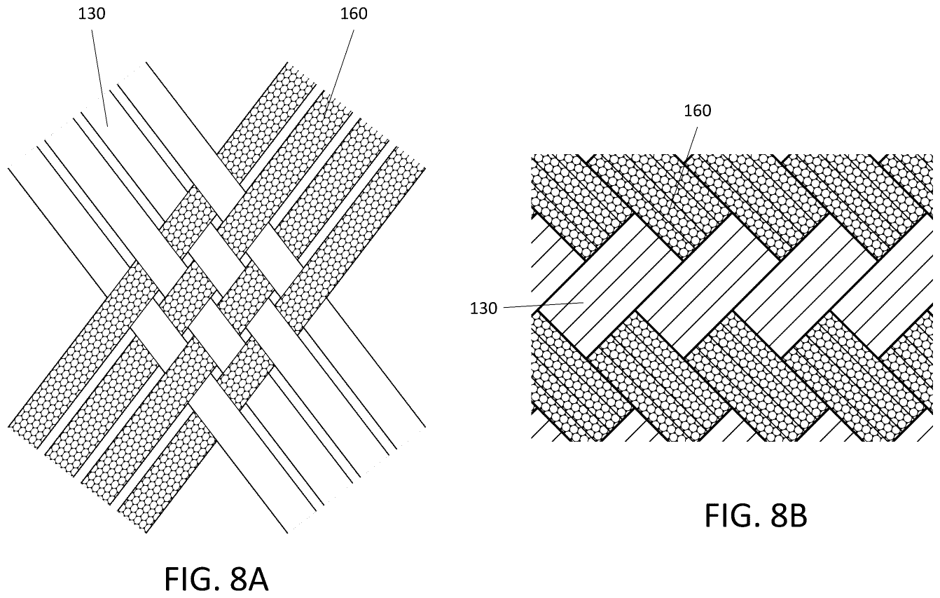

FIGS. 8A-8B show examples of weave patterns in which CNT fibers 160 are woven in and out of the lead conductors 130. FIG. 8A shows an example of a 1×1 plain weave pattern, while FIG. 8B shows an example of a 4×4 twill weave pattern. In one embodiment, the woven CNT fibers 160 and lead conductors 130 of these weave patterns may be individual strands. In another embodiment, the woven CNT fibers 160 and lead conductors 130 of these weave patterns may be a set of strands (e.g. 4) assembled together in a ribbon.

What is claimed is:

1. A system for providing neurostimulation to a patient, the system comprising:
a pulse generator including a housing, and
an implantable neurostimulation lead, wherein the implantable neurostimulation lead is configured to connect with the pulse generator;
wherein the pulse generator is configured to generate a plurality of electrical impulses for delivering a neurostimulation treatment to the patient through the neurostimulation lead when the lead is implanted at a target location;
wherein the implantable neurostimulation lead includes one or more conductors extending from a proximal end of the implantable neurostimulation lead to one or more neurostimulation electrodes disposed at or near a distal end of the implantable neurostimulation lead;
wherein the implantable neurostimulation lead includes an energy absorber comprising carbon nanotube material and extending substantially along the length of the one or more conductors; and
wherein the energy absorber is configured to absorb electromagnetic energy and conduct electromagnetic energy along the length of the implantable neurostimulation lead.

2. The system of claim 1, wherein the housing is connected to the energy absorber and serves as a component of the energy absorber.

3. The system of claim 2, wherein the housing is configured to dissipate the electromagnetic energy absorbed and conducted by the energy absorber.

4. The system of claim 1, wherein the energy absorber overlies the one or more electrodes.

5. A system for providing neurostimulation to a patient, the system comprising:
a pulse generator including a housing, and
an implantable neurostimulation lead, wherein the implantable neurostimulation lead is configured to connect with the pulse generator;
wherein the pulse generator is configured to generate a plurality of electrical impulses for delivering a neurostimulation treatment to the patient through the neurostimulation lead when the lead is implanted at a target location;
wherein the implantable neurostimulation lead includes a plurality of conductor wires extending from a proximal end of the implantable neurostimulation lead to one or more neurostimulation electrodes disposed at or near a distal end of the implantable neurostimulation lead;
wherein the implantable neurostimulation lead includes an energy absorber including a carbon nanotube material that is configured to shield the conductor wires from electromagnetic energy; and
wherein the energy absorber is configured to absorb electromagnetic energy and conduct electromagnetic energy along the length of the implantable neurostimulation lead.

6. The system of claim 5, wherein the housing is connected to the energy absorber and serves as a component of the energy absorber.

7. The system of claim 6, wherein the housing is configured to dissipate the electromagnetic energy absorbed and conducted by the energy absorber.

8. The system of claim 5, wherein the carbon nanotube material is a homogenous carbon nanotube material.

9. The system of claim 5, wherein the carbon nanotube material is a composite carbon nanotube material.

10. The system of claim 5, wherein the plurality of conductor wires are arranged in a coil extending along the length of the neurostimulation lead.

11. The system of claim 10, wherein the energy absorber is configured as an elongated wire embedded in the plurality of conductor wires.

12. The system of claim 5, wherein the energy absorber overlies the coil of conductor wires.

13. The system of claim 12, wherein the energy absorber includes a wire embedded in the plurality of electrode wires.

14. A system for providing neurostimulation to a patient, the system comprising:
a pulse generator including a housing, and
an implantable neurostimulation lead, wherein the implantable neurostimulation lead is configured to connect with the pulse generator;
wherein the pulse generator is configured to generate a plurality of electrical impulses for delivering a neurostimulation treatment to the patient through the neurostimulation lead when the lead is implanted at a target location;
wherein the implantable neurostimulation lead includes a plurality of conductors forming a conductor coil extending from a proximal end of the implantable neurostimulation lead to one or more neurostimulation electrodes disposed at or near a distal end of the implantable neurostimulation lead;
wherein the implantable neurostimulation lead includes an energy absorber comprised of a carbon nanotube material;
wherein the energy absorber is configured as at least one wire that is woven into the conductor coil; and
wherein the energy absorber is configured to absorb electromagnetic energy and conduct electromagnetic energy along the length of the implantable neurostimulation lead.

15. The system of claim 14, wherein the energy absorber and conductor coil are woven together in a plain weave pattern.

16. The system of claim 14, wherein the energy absorber and conductor coil are woven together in a twill weave pattern.

17. The system of claim 14, wherein the energy absorber comprises a plurality of wires.
